(19) United States
(12) Patent Application Publication
(10) Pub. No.: US 2022/0031798 A1
(43) Pub. Date: Feb. 3, 2022

(54) METHODS AND COMPOSITIONS FOR THE TREATMENT OF OPIOID DEPENDENCE AND FOR THE TREATMENT OF PAIN

(71) Applicant: Duke University, Durham, NC (US)

(72) Inventors: Ashwin Patkar, Durham, NC (US); Cynthia Kuhn, Durham, NC (US)

(73) Assignee: Duke University, Durham, NC (US)

(21) Appl. No.: 17/298,664

(22) PCT Filed: Dec. 4, 2019
(86) PCT No.: PCT/US19/64551

§ 371 Date: May 31, 2021

Related U.S. Application Data
(60) Provisional application No. 62/777,841, filed on Dec. 11, 2018.
(60) Provisional application No. 62/774,930, filed on Dec. 4, 2018.

(51) Int. Cl. A61K 38/07; A61P 25/36; A61P 25/04

(52) U.S. Cl. CPC A61K 38/07; A61P 25/04; A61P 25/36

(57) ABSTRACT

The present invention relates to methods of treating opioid dependence, enhancing the treatment of opioid dependence, treating opioid withdrawal, or alleviating one or more opioid withdrawal symptoms in a subject, preventing or reducing the likelihood of opioid dependence relapse in a subject treated for opioid dependence, reducing the vulnerability of a subject to develop opioid dependence in adulthood following opioid exposure during adolescence, or treating pain in a subject, comprising administering a therapeutically effective amount of a N-methyl-D-aspartate receptor (NMDA) partial agonist to the subject. The present invention further relates to compositions comprising an NMDA partial agonist for use with the aforementioned methods.

### CROSS REFERENCE TO RELATED APPLICATIONS

This application claims priority to U.S. Provisional Patent Application Ser. No. 62/774,930, filed Dec. 4, 2018, and U.S. Provisional Patent Application Ser. No. 62/777,841, filed Dec. 11, 2018, the contents of each of which are hereby incorporated by reference in their entirety.

### BACKGROUND OF THE INVENTION

### Field of the Invention

The present invention relates to methods of treating opioid dependence, enhancing the treatment of opioid dependence, treating opioid withdrawal, or alleviating one or more opioid withdrawal symptoms in a subject, preventing or reducing the likelihood of opioid dependence relapse in a subject treated for opioid dependence, reducing the vulnerability of a subject to develop opioid dependence in adulthood following opioid exposure during adolescence, or treating pain in a subject, comprising administering a therapeutically effective amount of a N-methyl-D-aspartate receptor (NMDA) partial agonist to the subject. The present invention further relates to compositions comprising an NMDA partial agonist for use with the aforementioned methods.

### Description of the Related Art

An increasing number of opioid overdose deaths have resulted from the rising availability of both prescription and nonprescription opioids. While current FDA-approved pharmacotherapies for the treatment of opioid dependence prevent overdose deaths, they maintain the patient in an opioid-dependent state throughout treatment or cause uncomfortable withdrawal side effects. These include methadone (an opioid agonist, which improves clinical outcomes, but maintains the opioid dependent state), buprenorphine (an opioid partial agonist, which can elicit withdrawal signs if patients use opioids during treatment), and naloxone (an opioid antagonist, which triggers withdrawal symptoms and so requires an opioid-abstinent period before treatment begins and can elicit marked withdrawal signs if patients attempt to overcome its blockade with high doses of opioids). Treatment requires long-term tapering with buprenorphine or methadone to attain drug-free abstinence, during which withdrawal symptoms can occur.

This therapeutic approach may be particularly problematic for the rising population of opioid-dependent adolescents. Few clinical trials have investigated efficacy of these treatments in adolescents, and prolonged pharmacotherapy could extend their period of opioid dependence for as long as or longer than their period of active opioid self-administration.

These limitations show a clear need for a non-opioid pharmacotherapy.

The important role of glutamate neurons in the acquisition, expression and relapse to opioid reward have generated interest in glutamate receptors as a potential pharmacologic target to treat opioid dependence, but success has been limited. Drugs that target the NMDA receptor have been proposed as alternatives to opioid-targeted pharmacotherapies. The NMDA receptor is involved in the maintenance of opioid dependence and its blockade may reverse the neuroadaptive changes induced by opioid dependence. Regulation of the NMDA receptor could accelerate treatment of opioid dependency (Glass et al. Exp Neurol. 2008; 210(2):750-761). Ketamine, an NMDA antagonist, has potential as a treatment, but has associated side effects and its known human abuse profile limits its potential utility (Liu Y. et al. Brain Res Bull. 2016; 126(Pt 1):68-73). The weak antagonist memantine has yielded mixed results in a small number of clinical trials with low patient numbers, which support the potential efficacy of such NMDA antagonists but has not demonstrated clear clinical efficacy (Bisaga, A et al., J. Substance Abuse Treatm. 5: 546-552 (2014); Elias et al. J. Subst. Abuse Treatment 107: 38-43 (2019)). Finally, the glycine-site modulator d-cycloserine facilitated CPP extinction and naloxone place aversion in animal models, but its clinical efficacy was limited by its short half-life in humans (Das R K, Kamboj S K. Biol Psychiatry. 2012; 72(11):e29-30; author reply e31-22).

Thus, there exists a need for improved pharmacotherapies to treat or enhance the treatment of opioid dependency and to prevent or reduce the likelihood of relapse.

### SUMMARY OF THE INVENTION

In a first aspect, the present invention provides a method of treating opioid dependence in a subject comprising administering a therapeutically effective amount of a N-methyl-D-aspartate receptor (NMDA) partial agonist to the subject.

In a second aspect, the present invention provides a method of enhancing the treatment of opioid dependence in a subject comprising administering a therapeutically effective amount of a NMDA partial agonist to the subject.

In a third aspect, the present invention provides a method of treating opioid withdrawal in a subject comprising administering a therapeutically effective amount of a NMDA partial agonist to the subject.

In a fourth aspect, the present invention provides a method of alleviating one or more opioid withdrawal symptoms in a subject comprising administering a therapeutically effective amount of a NMDA partial agonist to the subject.

In a fifth aspect, the present invention provides a method of preventing opioid dependence relapse in a subject treated for opioid dependence comprising administering a therapeutically effective amount of a NMDA partial agonist to the subject.

In a sixth aspect, the present invention provides a method of reducing the likelihood of opioid dependence relapse in a subject treated for opioid dependence comprising administering a therapeutically effective amount of a NMDA partial agonist to the subject.

In a seventh aspect, the present invention provides a method of reducing the vulnerability of a subject to develop opioid dependence in adulthood following opioid exposure during adolescence comprising administering a therapeutically effective amount of a NMDA partial agonist to the subject.

In an eighth aspect, the present invention provides a method of treating pain in a subject comprising administering a therapeutically effective amount of a NMDA partial agonist to the subject.

In certain embodiments of the above aspects of the invention, the NMDA partial agonist is rapastinel. In certain embodiments of the above aspects of the invention, the subject is a human, and in some embodiments, the human is an adolescent.

In a ninth aspect, the invention provides a composition comprising an NMDA partial agonist and a pharmaceutically acceptable carrier. In certain embodiments of this aspect of the invention, the NMDA partial agonist is rapastinel.

### BRIEF DESCRIPTION OF THE DRAWINGS

FIG. 1. Timeline for the studies described in Example 1.

FIG. 2. Withdrawal signs after morphine/naloxone challenge day 6 or 27 after 5 days of morphine. Results expressed as mean±SEM. N=10-15/morphine group and 6-10/saline group. *=p<0.05 or better relative to saline control. Adolescent and adult rats experienced comparable naloxone-precipitated withdrawal.

FIG. 3. Withdrawal signs after naloxone challenge on Day 9 after 3 days of saline, Ketamine or Rapastinel. Results are expressed as mean±SEM/N=24 for saline, 12 for ketamine, 14 for rapastinel. No sex differences were observed so data were combined. *=different from saline and ketamine. Rapastinel but not ketamine extinguished naloxone-precipitated withdrawal in adolescent rats.

FIG. 4. Withdrawal signs after naloxone challenge on day 9 after 5 days of morphine followed by 3 days of saline or rapastinel as in FIG. 3. Results expressed as mean±SEM. N=12-14/group. No main effect of sex was observed so data were combined. *=p<0.05 or better relative to saline control. Rapastinel extinguished naloxone-precipitated withdrawal comparably in adolescent and adult rats.

FIG. 5. Withdrawal signs after naloxone challenge on day 9 after 5 days of morphine followed by 3 days of saline or rapastinel as in FIGS. 3 and 4. Results expressed as mean±SEM. N=5-8 for females, 15-20 for males. *=p<0.05 or better relative to saline control. Rapastinel extinguished naloxone-precipitated withdrawal comparably in male and female rats.

FIG. 6. Schematic for withdrawal-precipitated hyperalgesia studies and schematic for von Frey test of pain sensitivity.

FIGS. 7A-7B. Hyperalgesia caused by morphine-withdrawal. Hyperalgesia habituation (FIG. 7A) and hyperalgesia—naloxone day 6 (FIG. 7B).

FIG. 8. Rapastinel treatment on Day 9 following treatment.

FIG. 9. Schematic of the post-synaptic neuron exposed to rapastinel.

FIG. 10. Controlled expression levels of GluR1 in the mPFC: morphine-treated rats have higher expression of GluR1 in the mPFC.

FIG. 11. Rapastinel blocks relapse to conditioned place preference (CPP).

### DETAILED DESCRIPTION OF THE INVENTION

For the purposes of promoting an understanding of the principles of the present disclosure, reference will now be made to particular embodiments of the invention and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. Embodiments recited as “including,” “comprising,” or “having” certain elements are also contemplated as “consisting essentially of and “consisting of those certain elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps and those that do not materially affect the basic and novel characteristic(s) of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

Unless otherwise defined, all technical terms used herein have the same meaning as commonly understood by one of ordinary skill in the art to which this disclosure belongs.

The inventors have discovered that NMDA partial agonists are effective in treating opioid dependence and mitigating symptoms of opioid withdrawal by bringing about a reduction in withdrawal symptoms.

Accordingly, in a first aspect, the present invention provides a method of treating opioid dependence in a subject comprising administering a therapeutically effective amount of a N-methyl-D-aspartate receptor (NMDA) partial agonist to the subject.

As used herein, “treatment,” “treating,” “therapy,” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “subject” and “patient” are used interchangeably and refer to both human and nonhuman animals. The term “nonhuman animals” includes all vertebrates, e.g., mammals and non-mammals, such as nonhuman primates, sheep, dog, cat, horse, cow, chickens, amphibians, reptiles, and the like. In some embodiments, the subject comprises a human suffering from an opioid dependency. In certain embodiments, the subject comprises an adolescent human suffering from an opioid dependency.

The term “effective amount” or “therapeutically effective amount” refers to an amount sufficient to effect beneficial or desirable biological and/or clinical results.

The N-methyl-D-aspartate receptor (NMDA) is found in nerve cells and is an ionotropic glutamate receptor. NMDA is activated when glutamate (or aspartate) and glycine (or D-serine) bind to it; binding of these co-agonists is required for efficient opening of the ion channel. NMDA partial agonists bind to the glutamate recognition site, the glycine recognition site, or to an allosteric site. Exemplary NMDA partial agonists include, but are not limited to, N-methyl-D-aspartic acid, 3,5-dibromo-L-phenylalanine, rapastinel, apimostinel, aminocyclopropanecarboxylic acid, cycloserine, HA-966, NYX-2925, and homoquinolinic acid.

In certain embodiments, the NMDA partial agonist used in the methods disclosed herein is rapastinel:

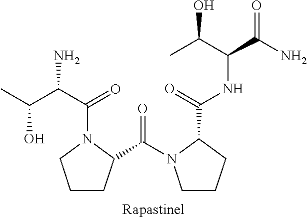

Rapastinel, an allosteric modulator of the glycine site of the NMDA receptor complex, has shown potential clinical efficacy against depression without the psychiatric side effects, abuse potential, or reinforcement of ketamine and other glutamate-targeted drugs. Unlike ketamine, rapastinel has not been shown to produce any negative side effects during treatment in animal models (Moskal et al., 2016). Rapastinel has been described as an agent that “stabilizes” NMDA receptor function, preventing excessive activation, but permitting activation when it is deficient. Rapastinel is a safe, fast-acting, long-lasting therapeutic free of psychoactive side effects like those seen with the similar therapy ketamine.

As disclosed herein, rapastinel improves overall withdrawal symptoms yet it does so without maintaining an opioid dependent state, which is a significant problem with current pharmacotherapies. Without wishing to be bound by any particular theory, the inventors believe that rapastinel suppresses withdrawal signs of opioid dependency and prevents/delays relapse by reversing neuroadaptations in NMDA receptor function.

In a second aspect, the invention provides a method of enhancing the treatment of opioid dependence in a subject comprising administering a therapeutically effective amount of a NMDA partial agonist to the subject.

As used herein, the term “enhancing” refers to the increase and/or further improvement of a treatment.

In a third and fourth aspect, respectively, the invention provides a method of treating opioid withdrawal in a subject comprising administering a therapeutically effective amount of a NMDA partial agonist to the subject, and a method of alleviating one or more opioid withdrawal symptoms in a subject comprising administering a therapeutically effective amount of a NMDA partial agonist to the subject.

As used herein, “opioid withdrawal symptoms” include, but are not limited to, hyperalgesia, insomnia, anhedonia, dilated pupils, diarrhea, and cravings for opioids. In certain embodiments of the fourth aspect of the invention, the opioid withdrawal symptom is hyperalgesia.

In a fifth and sixth aspect, respectively, the invention provides a method of preventing opioid dependence relapse in a subject treated for opioid dependence comprising administering a therapeutically effective amount of a NMDA partial agonist to the subject, and a method of reducing the likelihood of opioid dependence relapse in a subject treated for opioid dependence comprising administering a therapeutically effective amount of a NMDA partial agonist to the subject.

As used herein, “reducing the likelihood of opioid dependence relapse” means decreasing the percent chance of relapsing, increasing the time to relapse, or both.

In a seventh aspect, the invention provides a method of reducing the vulnerability of a subject to develop opioid dependence in adulthood following opioid exposure during adolescence comprising administering a therapeutically effective amount of a NMDA partial agonist to the subject.

As used herein, “adolescence” refers to the period of time between childhood and adulthood, generally beginning at puberty and ending at maturity into an adult. An “adolescent” as used herein refers to an animal in the stage of adolescence. For humans, an adolescent is between the ages of about 12 and about 21.

The inventors have also discovered that NMDA partial agonists, including rapastinel, have general analgesic properties and are therefore effective in the treatment of pain, i.e. they can be used to reduce or eliminate pain in a subject suffering therefrom, including a subject suffering from hyperalgesia.

Accordingly, in an eighth aspect, the invention provides a method of treating pain in a subject comprising administering a therapeutically effective amount of a NMDA partial agonist to the subject. In certain embodiments of the eighth aspect of the invention, the NMDA partial agonist is rapastinel.

The NMDA partial agonist (e.g., rapastinel) as used in the methods disclosed herein can be administered to a subject, either alone or formulated as a composition comprising the NMDA partial agonist and a pharmaceutically acceptable carrier or excipient, in an amount sufficient to induce an appropriate response (e.g., treat an opioid dependency).

Accordingly, in a ninth aspect, the invention provides a composition comprising an NMDA partial agonist and a pharmaceutically acceptable carrier.

A “pharmaceutically acceptable excipient,” “pharmaceutically acceptable carrier,” or “diagnostically acceptable excipient” includes but is not limited to, sterile distilled water, saline, phosphate buffered solutions, amino acid-based buffers, or bicarbonate buffered solutions. The excipient selected and the amount of excipient used will depend upon the mode of administration. It is also contemplated that the compositions of the invention or those used with the methods of the invention will contain certain pharmaceutically acceptable inert ingredients which are within the purview of one of skill in the art.

The NMDA partial agonists of the invention or those used with the methods of the invention may be administered as a composition comprising the inhibitor and one or more as pharmaceutically acceptable carriers, adjuvants, diluents, and/or excipients, and may be administered by any route known in the art, including, but not limited to, orally, intravenously, intraperitoneally, intramuscularly, intrathecally, subcutaneously, sublingually, buccally, rectally, vaginally, ocularly, otically, nasally, by inhalation, by nebulization, topically, and transdermally. In certain embodiments, the inhibitor is administered intravenously or intraperitoneally.

“Administration” as it applies to a human, primate, mammal, mammalian subject, animal, veterinary subject, placebo subject, research subject, experimental subject, cell, tissue, organ, or biological fluid, refers without limitation to contact of an exogenous ligand, reagent, placebo, small molecule, pharmaceutical agent, therapeutic agent, diagnostic agent, or composition to the subject, cell, tissue, organ, or biological fluid, and the like. “Administration” can refer, e.g., to therapeutic, pharmacokinetic, diagnostic, research, placebo, and experimental methods.

An “effective amount” as used herein means an amount which provides a therapeutic or prophylactic benefit. Effective amounts of an NMDA partial agonist (e.g., rapastinel) can be determined by a physician with consideration of individual differences in age, weight, extent of opioid dependency, and condition of the patient (subject). The optimal dosage and treatment regime for a particular patient can readily be determined by one skilled in the art of medicine by monitoring the patient for signs of withdrawal/dependency and adjusting the treatment accordingly. In a non-limiting example, the NMDA partial agonist may be dosed at 150-600 mg/weekly (e.g. intravenously), or any range or value within this range, e.g. 225-450 mg/weekly, 225 mg/weekly, or 450 mg/weekly.

An effective amount of a therapeutic agent is one that will decrease or ameliorate the symptoms normally by at least 10%, more normally by at least 20%, most normally by at least 30%, typically by at least 40%, more typically by at least 50%, most typically by at least 60%, often by at least 70%, more often by at least 80%, and most often by at least 90%, conventionally by at least 95%, more conventionally by at least 99%, and most conventionally by at least 99.9%.

An effective amount for a particular subject/patient may vary depending on factors such as the condition being treated, the overall health of the patient, the route and dose of administration and the severity of side effects. Guidance for methods of treatment and diagnosis is available (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

An effective amount of the NMDA partial agonist (e.g., rapastinel) described herein may be given in one dose, but is not restricted to one dose. Thus, the administration can be two, three, four, five, six, seven, eight, nine, ten, eleven, twelve, thirteen, fourteen, fifteen, sixteen, seventeen, eighteen, nineteen, twenty, or more, administrations of the NMDA partial agonist. Where there is more than one administration in the present methods, the administrations can be spaced by time intervals of one minute, two minutes, three, four, five, six, seven, eight, nine, ten, or more minutes, by intervals of about one hour, two hours, three, four, five, six, seven, eight, nine, ten, 11, 12, 13, 14, 15, 16, 17, 18, 19, 20, 21, 22, 23, 24 hours, and so on. In the context of hours, the term “about” means plus or minus any time interval within 30 minutes. The administrations can also be spaced by time intervals of 8 hours, 12 hours, one day, two days, three days, four days, five days, six days, seven days, eight days, nine days, ten days, 11 days, 12 days, 13 days, 14 days, 15 days, 16 days, 17 days, 18 days, 19 days, 20 days, 21 days, and combinations thereof. The present disclosure is not limited to dosing intervals that are spaced equally in time, but encompass doses at non-equal intervals, such as a priming schedule consisting of administration at 1 day, 4 days, 7 days, and 25 days, just to provide a non-limiting example.

A dosing schedule of, for example, once/day, twice/day, once/week, twice/week, three times/week, four times/week, five times/week, six times/week, seven times/week, once every two weeks, once every three weeks, once every four weeks, once every five weeks, and the like, is available for the invention. The dosing schedules encompass dosing for a total period of time of, for example, one week, two weeks, three weeks, four weeks, five weeks, six weeks, two months, three months, four months, five months, six months, seven months, eight months, nine months, ten months, eleven months, and twelve months.

Provided are examples of cycles of the above dosing schedules. The cycle can be repeated about, e.g., every seven days; every 14 days; every 21 days; every 28 days; every 35 days; 42 days; every 49 days; every 56 days; every 63 days; every 70 days; and the like. An interval of non-dosing can occur between a cycle, where the interval can be about, e.g., seven days; 14 days; 21 days; 28 days; 35 days; 42 days; 49 days; 56 days; 63 days; 70 days; and the like. In this context, the term “about” means plus or minus one day, plus or minus two days, plus or minus three days, plus or minus four days, plus or minus five days, plus or minus six days, or plus or minus seven days.

In certain embodiments, the NMDA partial agonist (e.g., rapastinel) according to the present disclosure may also be administered with one or more additional therapeutic agents (e.g., other opioid-withdrawal agents such as methadone, etc.). Methods for co-administration with an additional therapeutic agent are well known in the art (Hardman, et al. (eds.) (2001) Goodman and Gilman's The Pharmacological Basis of Therapeutics, 10th ed., McGraw-Hill, New York, N.Y.; Poole and Peterson (eds.) (2001) Pharmacotherapeutics for Advanced Practice: A Practical Approach, Lippincott, Williams & Wilkins, Phila., Pa.; Chabner and Longo (eds.) (2001) Cancer Chemotherapy and Biotherapy, Lippincott, Williams & Wilkins, Phila., Pa.).

Co-administration may refer to administration at the same time in an individual, but rather may include administrations that are spaced by hours or even days, weeks, or longer, as long as the administration of multiple therapeutic agents is the result of a single treatment plan. By way of example, administration of the NMDA partial agonist (e.g., rapastinel) of the present disclosure may comprise administering the NMDA partial agonist (e.g., rapastinel) of the present disclosure before, after, or at the same time as an additional therapeutic agent. This is not meant to be a limiting list of possible administration protocols.

### EXAMPLES

### Example 1: Rapastinel Significantly Enhances Recovery from Opioid Dependence

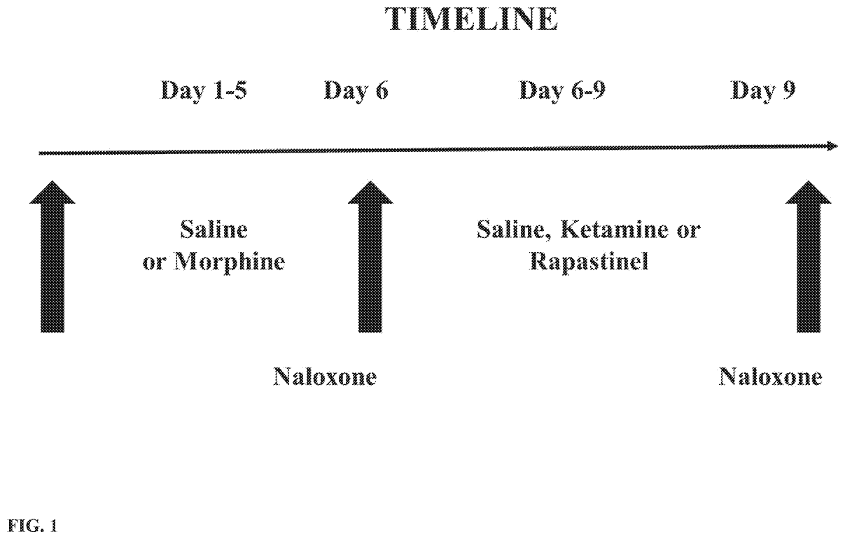

Male and female adolescent and adult Sprague-Dawley rats (PN 28-30 and PN 60-75) from Charles River Laboratories were treated with a 5-day, increasing dose morphine regimen (5 mg/kg bid, increasing 5 mg/kg/day to 25 mg/kg) to induce opioid dependency. Animals were group-housed in a 12/12 light/dark cycle with free access to water and standard lab chow. Agents were administered by intraperitoneal injection. In Study 1, animals received morphine (25 mg/kg) on day 6 followed 1 hour later by a naloxone challenge (1 mg/kg) and withdrawal behaviors were quantified as described by Gellert and Holtzman (J Pharmacol Exp Ther. 1978; 205(3):536-546). A second morphine/naloxone challenge was given 21 days later (study day 27). In Study 2 (see FIG. 1), male and female adolescent rats received the same morphine treatment, but on day 6 they received naloxone only (1 mg/kg) to precipitate withdrawal and withdrawal signs (wet dog shakes, diarrhea, mastication, salivation, ptosis and abnormal posture) were assessed as in Study 1. Then animals received saline, ketamine (1 mg/kg, bid) or rapastinel (5 mg/kg on alternate days). On day 9, animals received a second naloxone challenge (1 mg/kg) without a previous morphine treatment, and withdrawal signs were quantified to measure efficacy of ketamine and rapastinel. Study 3 compared the ability of rapastinel to accelerate the loss of opioid withdrawal signs in adult and adolescent rats and Study 4 compared rapastinel effects in male and female adults. Treatment results were analyzed by sequential 3-way (age×sex×treatment) and 1 way (treatment) ANOVA using NCSS followed by post-hoc Fishers LSD multiple comparison test to compare differences between groups. All experiments were approved by the Duke University IACUC and conducted in accordance with the NIH Guide for the Care and Use of Laboratory Animals.

The results of Study 1 indicated that naloxone elicited a robust withdrawal response on Day 6 (p<0.0001 for effect of treatment by ANOVA) but there was no effect of sex or age. The response to the second naloxone challenge on Day 27 was smaller but statistically significant (p<0.003 for effect of treatment) with significant effect of sex (p<0.04) and interaction of sex×age (p<0.0009). Female adults exhibited slightly higher withdrawal scores than male adults, while male and female adolescents had comparable withdrawal scores. In Study 2, no sex differences were observed so results were collapsed by sex. Rapastinel but not ketamine caused an enhanced loss of opioid withdrawal signs between day 6 and day 9 in adolescent males and females. Study 3 showed rapastinel was effective (p<0.002 for effect of treatment) but no age or sex differences were observed.

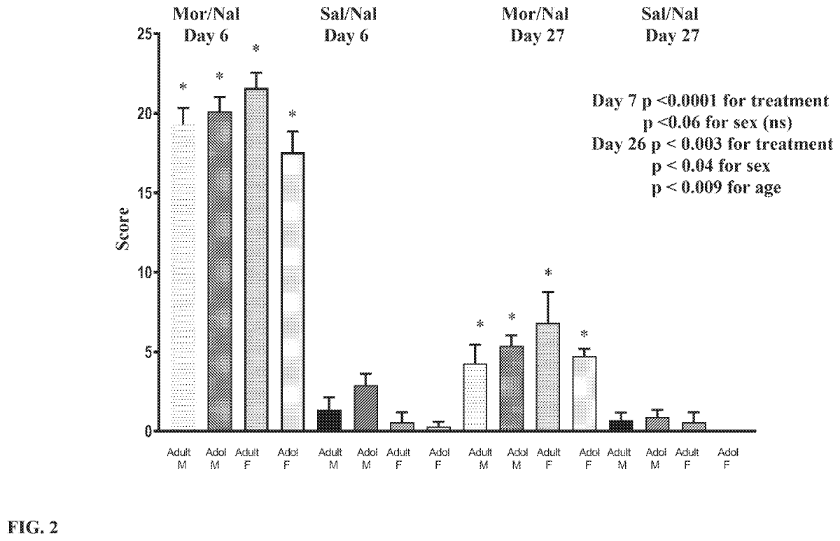

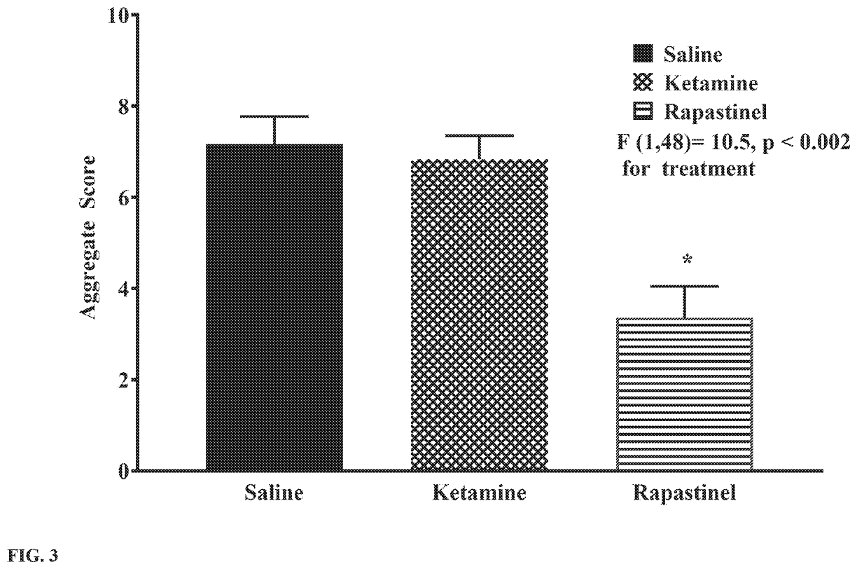

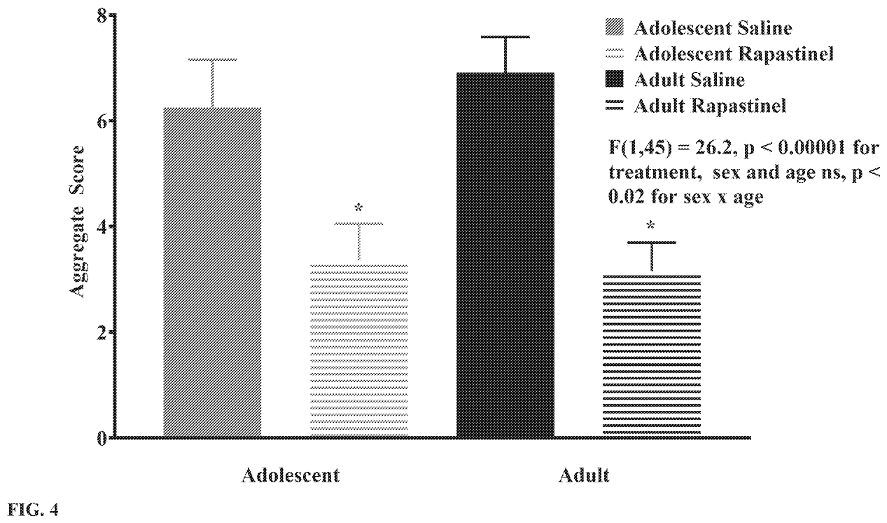

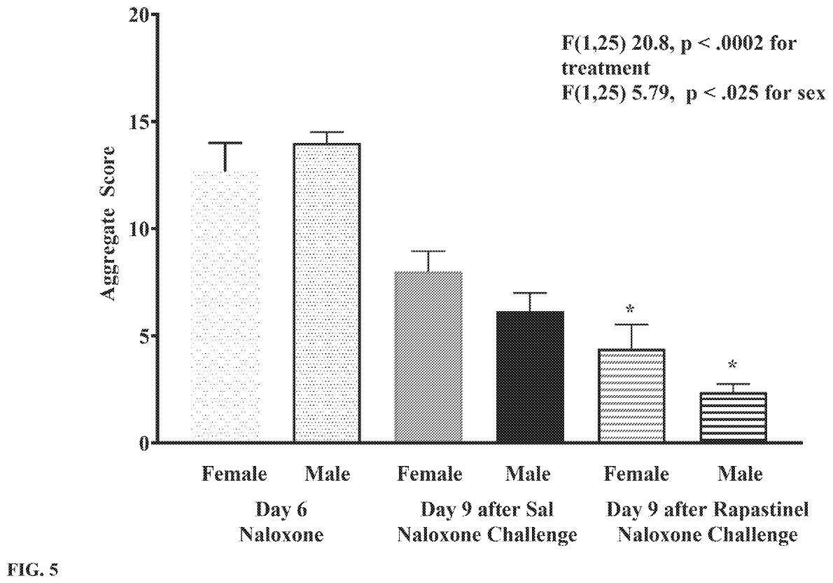

In summary, adolescent and adult rats exhibited comparable withdrawal signs from naloxone challenge after a brief (5 day) morphine treatment (see FIG. 2). Males and females exhibited comparable withdrawal signs (see FIG. 5). Rapastinel but not ketamine accelerated loss of withdrawal signs after termination of morphine treatment (see FIG. 3). Rapastinel accelerated extinction of opioid withdrawal at both ages (adolescents and adults) and in both sexes (see FIG. 4).

These studies show that opioid withdrawal signs are comparable early during withdrawal in adolescent and adult males and females. Females showed comparable withdrawal times on Day 6 and Day 9 but slightly exaggerated signs on Day 27, suggesting that they may show slightly slower loss of opioid dependence, but these effects were modest. Rapastinel significantly enhanced recovery from opioid dependence in both adolescent and adult rats, while ketamine was only marginally effective.

### Example 2: Rapastinel Improves Withdrawal-Precipitated Hyperalgesia

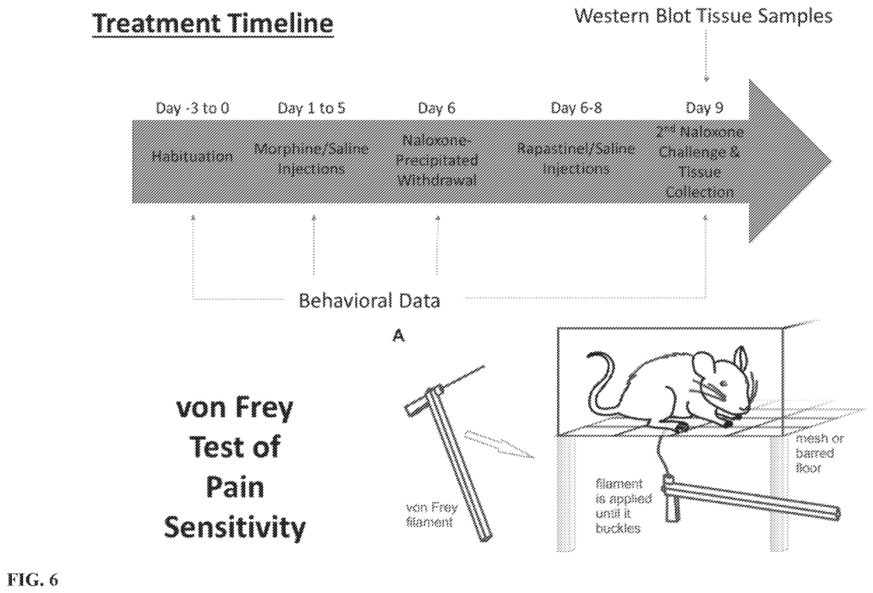

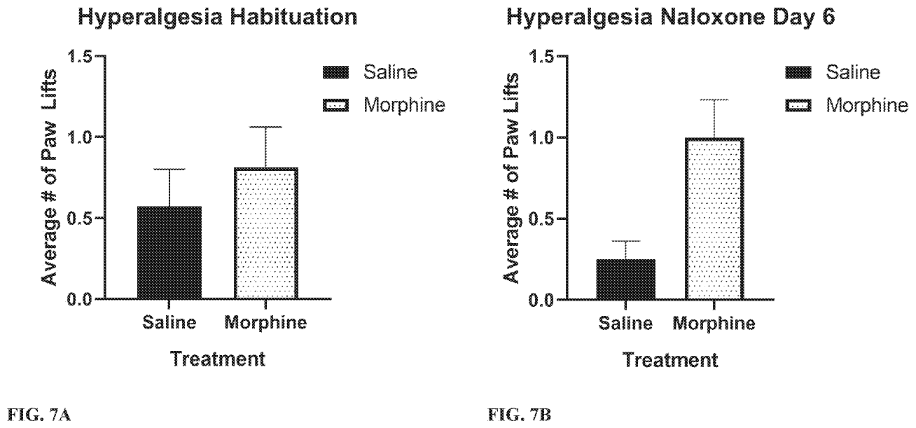

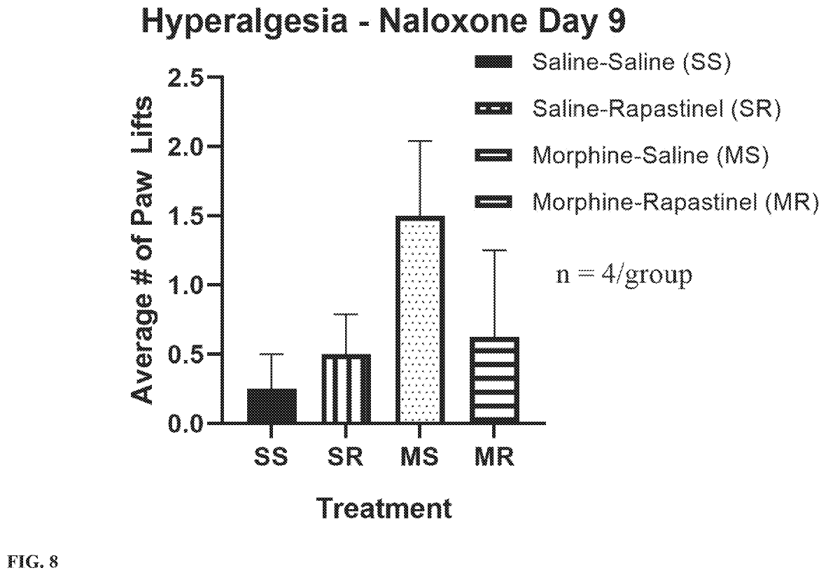

Rats were treated and pain sensitivity was measured in accordance with the schematics presented in FIG. 6. There was no difference in hyperalgesia caused by morphine withdrawal before treatment; there was significant expression of naloxone-precipitated hyperalgesia in morphine dependent animals. See FIG. 7. However, morphine-dependent rats treated with rapastinel trend towards a return to baseline (SS) hyperalgesia scores. See FIG. 8. Thus, rapastinel blunts hyperalgesia but does not alter baseline (SS) response, and is effective in the treatment of pain.

### Example 3: Rapastinel and the GluR₁ Receptor

The medial prefrontal cortex (mPFC) Brain Region is responsible for decision making in the context of reward (opioids) and penalty (withdrawal). Rats were treated with morphine in accordance with the methods of Example 1. The mPFC was dissected from rats at end of treatment and GluR₁ expression levels were measured with a Western Blot test.

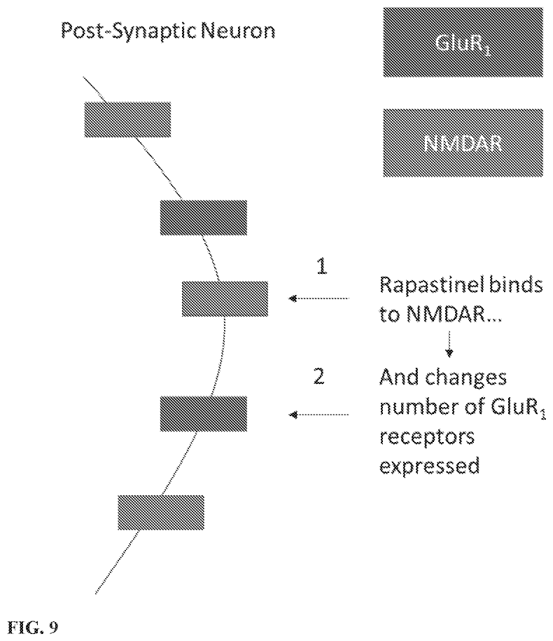

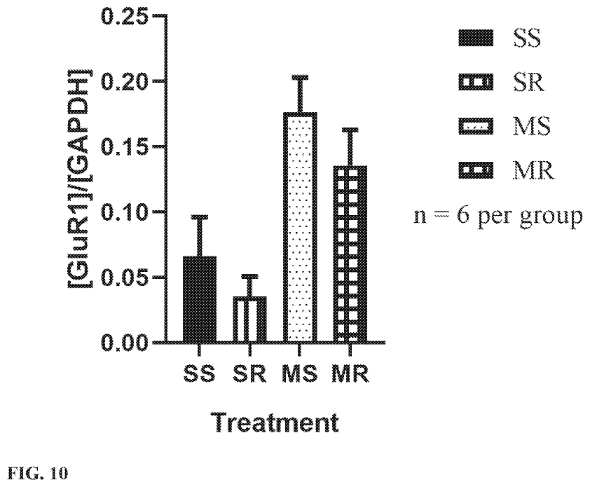

Rapastinel modulates GluR₁ expression as shown in FIG. 9. As shown in FIG. 10, morphine-treated rats, regardless of treatment, displayed significantly (p<0.001) higher GluR₁ expression in the mPFC. There was no significant effect of Rapastinel treatment on GluR₁ expression. Accordingly, rapastinel could be altering receptor expression differently in morphine-naïve and morphine-dependent rats, and might be treating hyperalgesia by a different mechanism.

### Example 4: Relapse to Morphine CPP

CPP and extinction were conducted in adult male and female rats with a biased CPP procedure adapted from Mueller et al. (Mueller et al., Behavior. Brain Res 136: 389-397(2002)). CPP is conducted in a two-sided chamber. Side 1 had white walls and plexiglass flooring, and Side 2 had black walls and plastic mesh flooring (preferred side).

Rats went through a conditioned place preference procedure involving daily pairing with saline and morphine (2.5 mg/kg) on opposite sides of the CPP apparatus for 4 days. On habituation day (day 1), animals were allowed to explore the whole apparatus for 30 minutes. On days 2-4, rats were restricted to Side 1 for morphine (2.5 mg/kg sc) and Side 2 for saline (alternated am and pm daily). On day 5 they received a test of conditioning in which they were allowed to explore the whole apparatus. For the next 4 days (days 6-9) they received daily saline or rapastinel (5 mg/kg) 30 minutes before twice daily extinction trials in which animals received saline on both sides of the apparatus. They received an extinction test in which they were allowed to explore the entire apparatus on day 10, and then a relapse test on day 11 in which they received a dose of saline or rapastinel, followed by saline or 2.5 mg/kg of morphine and were allowed to explore the apparatus.

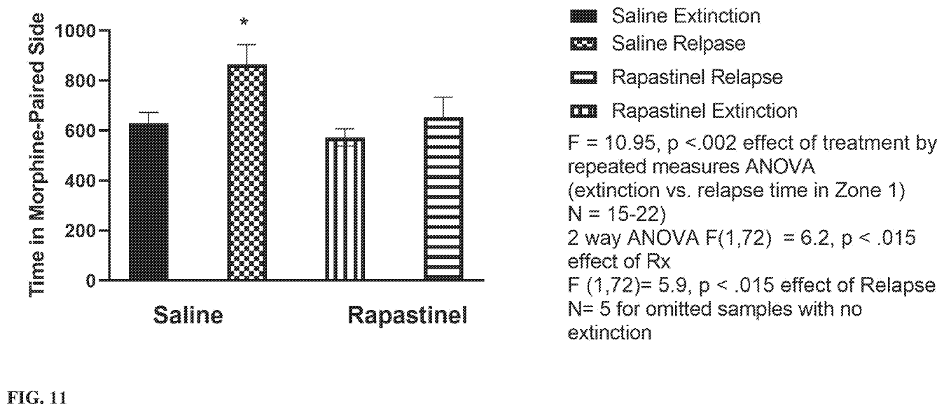

The data (see FIG. 11) shows the difference in the time spent on the drug-paired side on day 11 (the relapse test) compared to day 10 (the extinction trial). N=5 for rapastinel and 3 for saline. These data show that rapastinel treatment during extinction blunted morphine-induced relapse in the conditioned place preference test.

### Example 5: Vulnerability to Develop Opioid Dependence in Adulthood Following Opioid Exposure During Adolescence with and without Pharmacotherapy

Adolescent (PN 28) male and female rats are treated with saline or morphine (5 mg/kg days 1-2, 10 mg/kg days 3-4, 20 mg/kg days 5-6, 40 mg/kg days 7-8, 60 mg/kg days 9-10). This protocol has been shown to increase NMDA receptor subunit expression in nucleus accumbens. On day 38 (adolescent) they are given 1 mg/kg naloxone and drug-induced withdrawal signs are assessed. On day 38, they are evaluated for spontaneous withdrawal signs and then anxiety in a light/dark box, using standard methods for evaluating acute and sustained withdrawal. Animals from each treatment group (morphine, saline) are then subdivided into 2 cohorts, one of which receives rapastinel, and one saline (leading to final N=10/cell). Pharmacotherapy is initiated with saline, or rapastinel (10 mg/kg s.c.) every 5 days starting on PN 40. Spaced dosing is based on findings that rapastinel actions on depressive like behaviors and memory in rats persevere for many days even though this compound has a very short life. On PN 41, animals receive another saline or naloxone challenge and light/dark test on PN 42 to evaluate withdrawal signs in the presence of pharmacotherapy. On PN 60, animals enter the 13-day CPP/extinction trial.

Animals undergo morphine CPP and extinction/relapse after 20 days of saline or rapastinel treatment. This allows morphine-treated animals to recover from most withdrawal signs that could facilitate establishment of morphine CPP. Animals have active drug present throughout the CPP/extinction procedure. CPP is assessed with a trial on day 5 of the procedure. Animals then enter an 8-day extinction trial, and a final probe with morphine to assess “relapse” after extinction. Animals are used as their own controls, allowing within-animal comparison to determine if intensity of initial withdrawal correlates with treatment efficacy and minimization of the number of animals used. Animals receive a final anxiety test on PN 95 and then are euthanized; brains are banked for future analysis of NMDA receptor subunits.

### Example 6: NMDA Receptor Function in the Nucleus Accumbens

Animals receive either morphine or saline for 10 days as in Example 5. On day 11 acute brain slices are prepared containing the nucleus accumbens (NAcc). Using whole-cell patch clamp techniques, evoked monosynaptic glutamate receptor-mediated responses are recorded from medium spiny neurons in NAcc shell in the presence of picrotoxin (75 μM) or bicuculline (10 μM).

Glutamatergic synaptic currents are evoked using a locally-placed stimulus electrode. Input/output curves are generated with 5 increasing stimulus steps (initial intensity typically 10 to 150 μA, 0.01 μsec duration) delivered to the slice at a frequency of 0.33 hz. Initially neurons are voltage-clamped at both −70 and +40 mV to record both AMPA receptor- and NMDA receptor-mediated currents, respectively. AMPA-mediated response is measured at the peak of the current trace while NMDA-mediated responses are measured 60 msec after the electrical stimulation, as the short transient AMPA currents become rectified while held above 0 mV and essentially disappear 35 msec after the electrical stimulation. This allows for measuring differences in both the absolute magnitude of NMDA responses and the magnitude relative to AMPA responses in the same neurons. DNQX (20 μM) is then added to the superfusate to block AMPA-mediated responses. Using a fixed stimulus intensity, isolated NMDA receptor mediated responses are recorded while the holding voltage is increased from −90 to +50 in 10 mV increments to determine if prior morphine treatment alters the voltage dependence of NMDA receptors. Rising and decaying kinetics of NMDA receptor-mediated currents are additionally measured.

In a separate cohort, animals receive either morphine or saline×10 days as above, followed by 20 or 55 days of either saline or rapastinel to evaluate NMDA receptor function at the time of relapse assessment as in Example 5. The electrophysiological recordings are conducted as above.

### Example 7: Specific Methods

Rats and drug treatment: Male and female adolescent (PN 28) CD-1 Sprague Dawley rats from Charles River Labs in Raleigh are used. They are housed in self-ventilated and ad/lib Purina 5002 rodent chow and water. For euthanasia, rats are anesthetized with urethane (3 g/kg). Estrous cycles are noted at PN 38 and 73 by vaginal lavage.

Light-Dark Box: Rats are placed in a locomotor box, half of which is darkened, for 10 minutes and locomotion is captured by photocells. Time and distance in light and latency to enter light is measured as previously validated for adolescents.

Opioid withdrawal: Animals are treated with saline or naloxone as described. Ten minutes later they are scored by a modification of the Gellert/Holtzman scale for wet dog shakes, diarrhea, mastication, salivation, ptosis and abnormal posture.

CPP and extinction are assessed with a modification of procedure of Mueller et al. Animals are treated in a two-sided box that is used for anxiety testing. The anxiety test (10 minutes) serves as a single pre-exposure test of side bias. The next day rats receive 4 saline and 4 morphine (5 mg/kg) pairings (alternated am and pm daily) for each of 4 days. On day 5, animals receive the CPP test in which they can explore the apparatus freely. For the next 8 days, they receive saline pairings in both sides once daily. A final CPP trial is conducted on day 13 in which animals receive saline or morphine (2.5 mg/kg) and can explore the apparatus freely. Data are expressed as time on drug-paired side.

1. (canceled)

2. (canceled)

3. (canceled)

4. A method of treating hyperalgesia in a subject, the method comprising: administering a therapeutically effective amount of a NMDA partial agonist to the subject.

5. (canceled)

6. (canceled)

7. (canceled)

8. A method of reducing the vulnerability of a subject to develop opioid dependence in adulthood following opioid exposure during adolescence, the method comprising: administering a therapeutically effective amount of a NMDA partial agonist to the subject.

9. (canceled)

10. The method according to claim 4 wherein the NMDA partial agonist is rapastinel.

11. The method according to claim 4 wherein the subject is a human.

12. The method according to claim 11 wherein the human is an adolescent.

13. A composition comprising an NMDA partial agonist and a pharmaceutically acceptable carrier.

14. The composition of claim 13 wherein the NMDA partial agonist is rapastinel.

15. (canceled)

16. (canceled)

17. (canceled)

18. (canceled)

19. (canceled)

20. (canceled)

21. (canceled)

22. (canceled)

23. (canceled)

24. (canceled)

25. (canceled)

26. (canceled)

27. The method according to claim 11, wherein the human is an adult.

28. The method according to claim 4, wherein the subject has an opioid dependence.

29. The method according to claim 4, wherein the NMDA partial agonist comprises N-methyl-D-aspartic acid, 3,5-dibromo-L-phenylalanine, apimostinel, aminocyclopropanecarboxylic acid, cycloserine, HA-966, NYX-2925, or homoquinolinic acid.

30. The method according to claim 4, further comprising: repeating the administering step.

31. The method according to claim 4, further comprising: preventing opioid dependence relapse.

32. The method according to claim 8, wherein the NMDA partial agonist is rapastinel.

33. The method according to claim 8, wherein the NMDA partial agonist comprises N-methyl-D-aspartic acid, 3,5-dibromo-L-phenylalanine, apimostinel, aminocyclopropanecarboxylic acid, cycloserine, HA-966, NYX-2925, or homoquinolinic acid.

34. The method according to claim 8, further comprising: repeating the administering step.

35. The method of according to claim 8, further comprising: treating hyperalgesia in the subject.

36. The method according to claim 8, further comprising: preventing opioid dependence relapse.

37. The method according to claim 8, wherein the subject is a human.

38. The method according to claim 37, wherein the human is an adolescent.

39. The method according to claim 37, wherein the human is an adult.
